(19) United States
(12) Patent Application Publication
(10) Pub. No.: US 2025/0194959 A1
(43) Pub. Date: Jun. 19, 2025

(54) TARGETED TRAINING FOR RECIPIENTS OF MEDICAL DEVICES

(71) Applicant: Cochlear Limited, Macquarie University, NSW (AU)

(72) Inventors: Naomi Croghan, Centennial, CO (US); Harish Krishnamoorthi, Englewood, CO (US); Sara Ingrid Duran, Denver, CO (US); Christopher Joseph Long, Centennial, CO (US)

(21) Appl. No.: 19/056,003

(22) PCT Filed: Aug. 18, 2023
(86) PCT No.: PCT/IB2023/058294

§ 371 Date: Feb. 18, 2025

Related U.S. Application Data
(60) Provisional application No. 63/400,805, filed on Aug. 25, 2022.

(51) Int. Cl. A61B 5/12; A61B 5/372; A61N 1/04; G16H 20/40

(52) U.S. Cl. CPC A61B 5/12; A61B 5/372; A61N 1/04; G16H 20/40

(57) ABSTRACT

Presented herein are techniques for presenting recipients with targeted training based upon, for example, a recipient's “predicted” or “estimated” sensitivity and a recipient's “behavioral” or “subjective” sensitivity. The predicted sensitivity can be determined, for example, from an objective measure and the recipient's behavioral sensitivity can be determined from a behavioral (subjective) response to a stimulus. For cochlear implant recipients, the predicted/estimated sensitivity can be an estimated auditory sensitivity and the behavioral sensitivity can be a behavioral (subjective) auditory sensitivity.

### BACKGROUND

### Field of the Invention

The present invention relates generally to training of recipients of wearable or implantable medical devices, such as auditory training of cochlear implant recipients.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In some aspects, the techniques described herein relate to a method including: determining, from at least one objective measure, an estimated auditory sensitivity of a recipient of a hearing device; determining, from at least one subjective measure, a behavioral auditory sensitivity of the recipient; and providing an auditory training recommendation based upon the estimated auditory sensitivity and the behavioral auditory sensitivity.

According to other aspects, the techniques described herein relate to a method including: determining neural health of a recipient; estimating a predicted sensory sensitivity for the recipient based upon the neural health; estimating a behavioral sensory sensitivity of the recipient; comparing the behavioral sensory sensitivity of the recipient with the predicted sensory sensitivity; and providing targeted sensory training based upon the comparing.

According to still other aspects, the techniques described herein relate to one or more non-transitory computer readable storage media including instructions that, when executed by a processor, cause the processor to: obtain, from at least one objective measure, an estimated auditory sensitivity of a recipient of a hearing device; obtain a behavioral auditory sensitivity of the recipient; determine a difference between the estimated auditory sensitivity and the behavioral auditory sensitivity; and provide an auditory training recommendation based upon the difference between the estimated auditory sensitivity and the behavioral auditory sensitivity.

In some aspects, the techniques described herein relate to an apparatus including: one or more memories; and one or more processors configured to: determine, from data stored in the one or more memories indicative of at least one objective measure, an estimated auditory sensitivity of a recipient of a hearing device; determine, from data stored in the one or more memories indicative of at least one subjective measure, a behavioral auditory sensitivity of the recipient; and provide an auditory training recommendation based upon the estimated auditory sensitivity and the behavioral auditory sensitivity.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a flowchart illustrating a first process flow implementing the targeted training techniques of this disclosure;

FIG. 3 is a flowchart illustrating a second process flow implementing the targeted training techniques of this disclosure;

FIG. 4 is a schematic diagram of an arrangement of electrodes and neurons illustrating a neural health map determination utilized in the targeted training techniques of this disclosure;

FIG. 5 is a schematic diagram illustrating a neural health map utilized in the targeted training techniques of this disclosure;

FIG. 6 is a schematic diagram illustrating a cochlear implant fitting system with which aspects of the techniques presented herein can be implemented;

FIG. 7 is a schematic diagram illustrating an implantable stimulator system with which aspects of the techniques presented herein can be implemented;

FIG. 8 is a schematic diagram illustrating a vestibular stimulator system with which aspects of the techniques presented herein can be implemented; and

FIG. 9 is a schematic diagram illustrating a retinal prosthesis system with which aspects of the techniques presented herein can be implemented.

### DETAILED DESCRIPTION

Recipients of wearable or implantable medical devices can experience varying outcomes from use of those devices. For example, individual cochlear-implant recipients can vary in their neural survival patterns, electrode placement, neurocognitive abilities, etc. Targeted recipient training, such as targeted auditory training for cochlear implant recipients, can help maximize outcomes for different recipients. Unfortunately, it can be difficult to determine which recipients will benefit the most from additional rehabilitation and what kind of training will have the greatest impact. Due at least in part to this lack of personalization, outcomes across groups of recipients (e.g., hearing outcomes of cochlear implant recipients) are highly variable, and some individuals can not achieve their full potential of performance with the device. Accordingly, presented herein are techniques for presenting recipients with targeted training based upon, for example, a recipient's “predicted” or “estimated” sensitivity and a recipient's “behavioral” or “subjective” sensitivity. The predicted sensitivity can be determined, for example, from an objective measure and the recipient's behavioral sensitivity can be determined from a behavioral (subjective) response to a stimulus. For cochlear implant recipients, the predicted/estimated sensitivity can be an estimated auditory sensitivity and the behavioral sensitivity can be a behavioral (subjective) auditory sensitivity.

For example, for a cochlear implant recipient, the predicted/estimated sensitivity can be determined from one or more objective measures, such as a Neural Response Telemetry (NRT) measure and an electrode distance measurement. In particular, a neural-health map can be derived from the NRT measure and the electrode distance measurement to determine the “estimated auditory sensitivity” of the recipient to a subjective test, such as a behavioral auditory test. The behavioral auditory test is performed and the results, referred to as the “behavioral auditory sensitivity” can be evaluated against the estimated auditory sensitivity. The results of the evaluation can, in turn, be used to determine auditory training for the recipient.

In particular, if the behavioral auditory sensitivity does not reach the expected level of performance (e.g., the actual/determined behavioral auditory sensitivity is below the estimated auditory sensitivity), then one type of individualized and targeted auditory training plan can be prescribed for the recipient based on the difference. On the other hand, if the behavioral auditory test meets or exceeds the expected level of performance (e.g., the actual/determined behavioral auditory sensitivity is the same as, or above, the estimated auditory sensitivity), then another type of individual and targeted auditory training plan can be prescribed in which one or more forms of auditory training are decreased or omitted altogether. Accordingly, the disclosed techniques can provide clear guidance for auditory rehabilitation, reducing formerly extensive training for recipients who do not need it (thereby saving time and financial investment) and guiding efficient training and device adjustment for poor performers.

According to specific example embodiments, the objective test can take the form of an electroencephalogram measurement, an electrocochleography measurement, a blood test, a measure of an age of the recipient, a measure of a length of time the recipient has experienced hearing loss, an electrode placement imaging test, an NRT measurement test and/or others known to the skilled artisan. Combinations of the objective tests can also be used. The subjective tests used can take the form of iterative speech testing, speech recognition tests, phoneme discrimination tests, spectral ripple tests, modulation detection tests, pitch discrimination tests, or others known to the skilled artisan. Similar to the objective tests, combinations of the above-described subjective tests can be used in the disclosed techniques without deviating from the inventive concepts of this disclosure. With respect to the auditory training prescribed according to the disclosed techniques, recipients can be prescribed auditory training that can include syllable counting training, word emphasis training, phoneme discrimination and identification training, frequency discrimination training, text following exercises, time compressed-speech recognition exercises, complex speech passage comprehension exercises, and others known to the skilled artisan.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

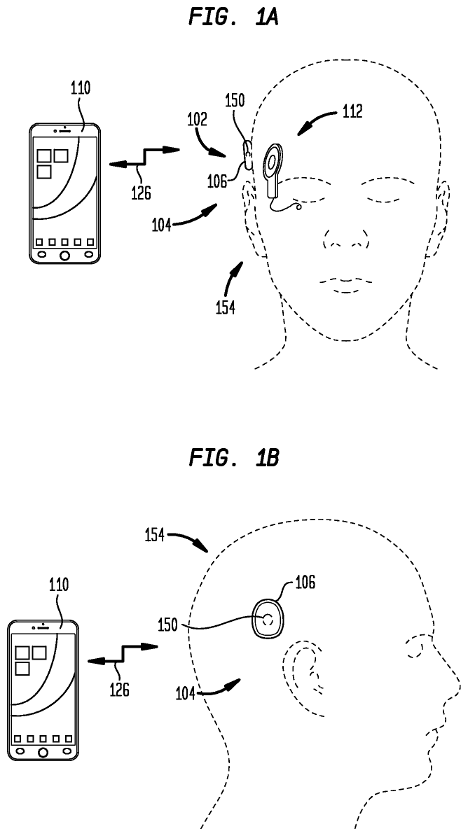

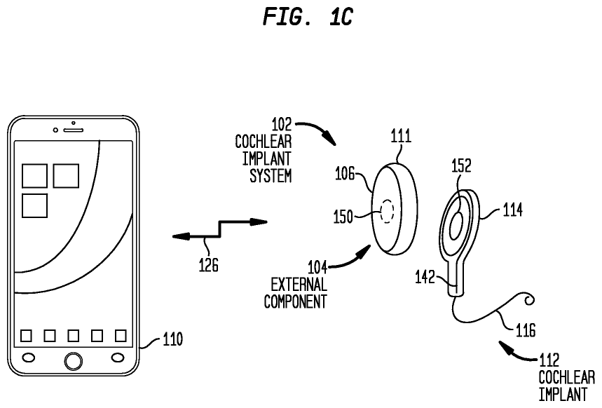

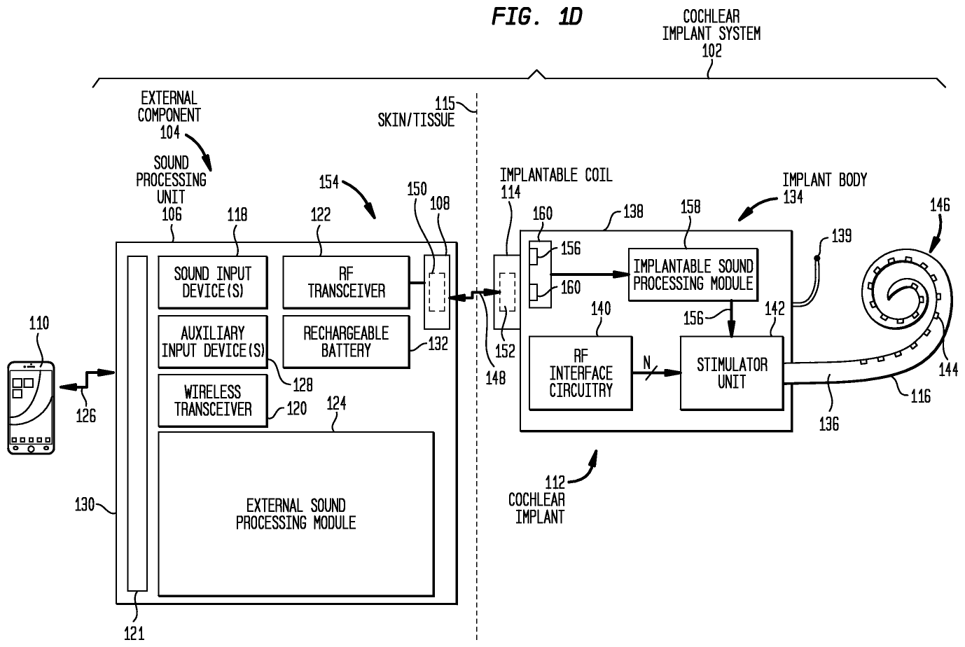

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 121 (e.g., for communication with the external device 110). However, it is to be appreciated that one or more input devices can include additional types of input devices and/or less input devices (e.g., the wireless short range radio transceiver 121 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

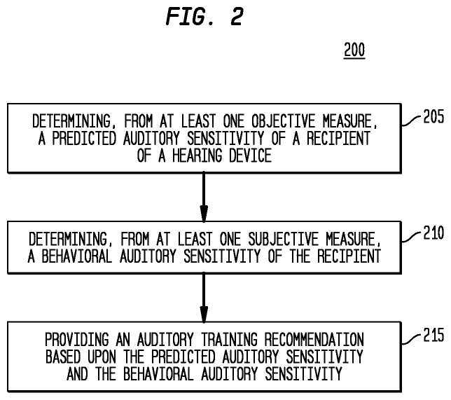

As noted above, the techniques of this disclosure can be used to prescribe or recommend targeted sensitivity (e.g., auditory) training for a recipient of a medical device, such as an auditory prosthesis like those described above with reference to FIGS. 1A-D. Accordingly, illustrated in FIG. 2 is a flowchart 200 providing a process flow for implementing the techniques of this disclosure. For ease of explanation, the example of FIG. 2 is described with specific reference to auditory sensitivity and training. However, it is to be appreciated that these techniques can also be utilized outside of auditory training.

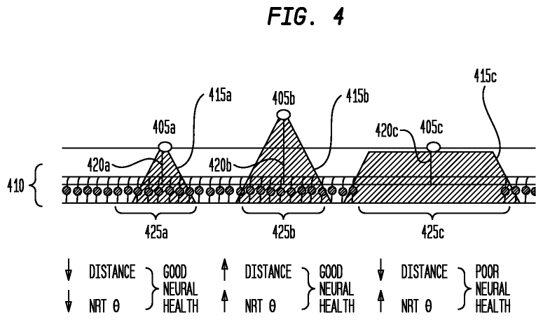

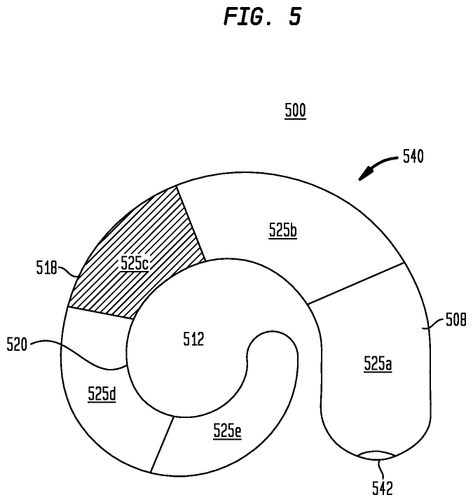

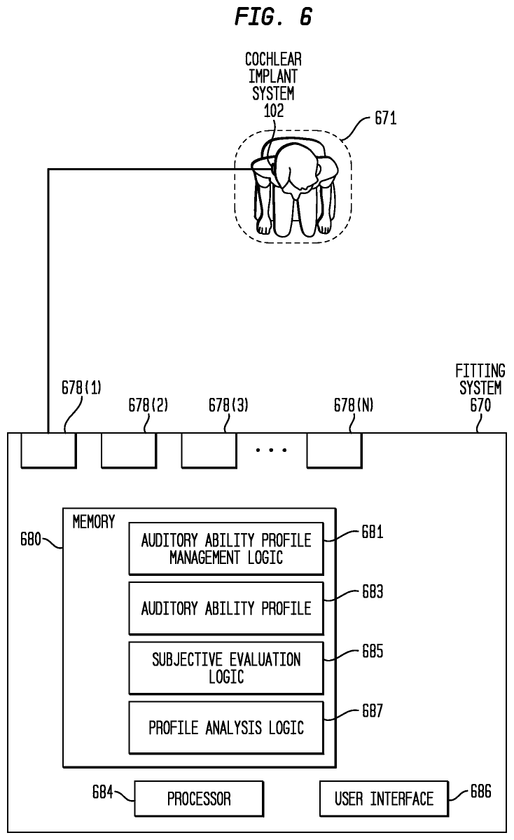

Flowchart 200 begins with operation 205 in which a predicted/estimated auditory sensitivity of a recipient of a hearing device (e.g., auditory prosthesis) is determined from at least one objective measure. Examples of the objective measure can include an NRT measurement, a measure of electrode distance to an associated neuron, an electroencephalogram measurement, an electrocochleography measurement, a blood test, a measure of an age of the recipient, a measure of a length of time the recipient has experienced hearing loss, or others known to the skilled artisan. Operation 205 can also include taking multiple measurements, of the same or different type, to determine the estimated auditory sensitivity of the recipient. For example, as described in detail below with reference to FIGS. 4 and 5, objective measures in the form of NRT measurements combined with electrode distance measurements can be used to determine a level of neural health of a recipient. Based upon the neural health determination, which can take the form of a neural health map for the recipient, an estimated auditory sensitivity can be determined for the recipient. According to other examples, an objective measure of the recipient's age can be combined with an objective measure of how long the recipient has experienced hearing loss to determine the estimated auditory sensitivity. These are just a few examples of the types of objective measurements, taken alone or in combination with additional and/or different objective measurements, that can be used in embodiments of operation 205.

In operation 210, a behavioral or subjective auditory sensitivity of the recipient is determined from at least one subjective measure. As used herein, a subjective measure (sometimes referred to herein as a behavioral measure) refers to a measure in which a user provides a behavioral response to some form of stimulus. For example, the subjective measure can be embodied as an iterative speech test of the recipient's hearing or auditory perception. Other forms of subjective measures can include speech recognition tests, phoneme discrimination tests, spectral ripple tests, modulation detection tests, pitch discrimination tests, and others known to the skilled artisan. While flowchart 200 illustrates operation 210 as following operation 205, this order can be switched or operations 205 and 210 can take place concurrently without deviating from the disclosed techniques.

Next, in operation 215, an auditory training recommendation is provided based upon the estimated auditory sensitivity and the behavioral or subjective auditory sensitivity. Certain embodiments of operation 215 can compare the estimated auditory sensitivity determined in operation 205 to the behavioral or subjective auditory sensitivity determined in operation 210. Differences between these sensitivities can determine the specific auditory training recommendation provided in operation 215. For example, if the behavioral or subjective auditory sensitivity outcome meets or exceeds the estimated auditory sensitivity, then no additional training is prescribed. Furthermore, if the recipient is already executing a training prescription, the prescription provided by operation 215 can include an option to discharge the recipient from the training. On the other hand, if the behavioral or subjective auditory sensitivity is slightly poorer than the estimated auditory sensitivity, then minimal training is prescribed, and if the behavioral or subjective auditory sensitivity is much poorer than the estimated auditory sensitivity, then greater training is prescribed.

According to one specific example, a behavioral phoneme test is used to measure auditory sensitivity in operation 210, and the outcome result is poorer than the estimated auditory sensitivity threshold determined in operation 205. More specifically, the phoneme confusion matrix from the behavioral test shows minor confusions between voiceless and voiced consonants. Accordingly, the targeted auditory training prescription provided in operation 215 recommends a “voiceless vs. voiced consonants in words and phrases” exercise to be conducted 1 time per day for 3 days. The behavioral phoneme test can be repeated after completion of the auditory training exercises to evaluate the effect of the targeted training.

According to another specific example, a sentence recognition task is used to measure auditory sensitivity in operation 210. The outcome result is below (poorer than) the estimated auditory sensitivity threshold determined in operation 205. Furthermore, the analysis from the behavioral test shows incorrect sentence length identification and significant vowel and consonant confusions. The targeted auditory training prescription provided in operation 215 can then recommend a “word or phrase length identification” exercise to be conducted 1 time per day for 3 days, followed by five different phoneme discrimination tasks to be conducted in order of ascending difficulty, with each task conducted 2 times per day for 3 days. The sentence recognition task is repeated after completion of the auditory training exercises to evaluate the effect of the targeted training.

The auditory training recommended in operation 215 can fall into different categories of training, including syllable counting training, word emphasis training, phoneme discrimination and identification training, frequency discrimination training, text following exercises, time compressed-speech recognition exercises, complex speech passage comprehension exercises, and others known to the skilled artisan. According to specific examples, syllable counting exercises can have the recipient identify the number of syllables or the length of words or phrases in testing data sets, while word emphasis exercises have the recipient identify where stress is being applied in the words of a training data set. Phoneme discrimination and identification tests can take many forms, including:
Contrasting vowel formant discrimination exercises;
Contrasting vowel length discrimination exercises;
Vowel identification in words and sentences exercises;
Consonant pattern identification exercises;
Word identification with common consonant confusions exercises;
Voiceless vs. voiced consonants in words and phrases exercises; and
Manner of articulation in words and phrases exercises, among others.

Frequency discrimination training can include pitch ranking exercises and/or high and low frequency phrase identification exercises. Depending on the results of operations 205 and 210, operation 215 can recommend or prescribe one or more of the above above-described exercises to be conducted over a specified period of time.

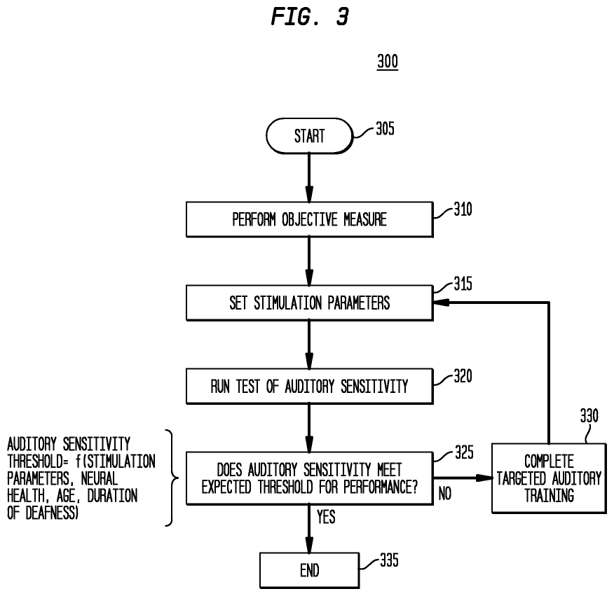

Flowchart 200 includes operations 205-215, but more or fewer operations can be included in methods implementing the disclosed techniques, as will become clear from the following discussion of additional examples of the disclosed techniques, including flowchart 300 of FIG. 3. Flowchart 300 implements a process flow according to the techniques of this disclosure that includes operations for setting stimulation parameters for an implantable medical device, such as a cochlear implant. The process flow begins in operation 305 and continues to operation 310 where an objective measure is made. Operation 310 can be analogous to operation 205 of FIG. 2. According to more specific embodiments of the disclosed techniques, operation 310 can be embodied as the generation of a neural health map, as described in detail below with reference to FIGS. 4 and 5.

In operation 315, stimulation parameters are set for the implantable medical device. With respect to a cochlear implant, the stimulation parameters can include the degree of focusing for focused multipolar stimulation by the cochlear implant, the assumed spread of excitation for the cochlear implant, a number of active electrodes, a stimulation rate, stimulation level maps for both threshold and comfortable loudness, frequency allocation boundaries, and others known to the skilled artisan.

In operation 320, a test is run to determine the behavioral auditory sensitivities of the recipient. Operation 320 can be analogous to operation 210 of FIG. 2, and the tests run in operation 320 can be one or more of an iterative speech test, a speech recognition test, a phoneme discrimination test, a spectral ripple tests a modulation detection test, a pitch discrimination test, or others known to the skilled artisan. Next, in operation 325, a determination is made as to whether the behavioral sensitivities determined in operation 320 meet or exceed an expected or predicted threshold for performance. Thresholds for performance used in the determination of operation 325 can be determined from the objective measure of operation 310. Furthermore, the expected or estimated auditory sensitivity can be a function of the stimulation parameters in combination with the objective measure. Accordingly, as illustrated in FIG. 3, the predicted or expected auditory sensitivity threshold can be a function of the stimulation parameters in combination with one or more of neural health, recipient age, duration of hearing loss, type of hearing loss, the results of an electroencephalogram, the results of an electrocochleograph, and/or the results of a blood test. In other words, the predicted or expected auditory sensitivity threshold of operation 320 can be derived from objective measures of a recipient's auditory sensitivity.

If the auditory sensitivity determined in operation 320 fails to meet or exceed the expected auditory sensitivity threshold determined in operation 325, auditory training can be prescribed for the recipient, which is performed by the recipient in operation 330. Upon completion of the auditory training, the process flow of flowchart 300 can return to operation 315, and the process flow will repeat until the auditory sensitivity determined in operation 320 meets or exceeds the expected auditory sensitivity threshold in operation 325, at which time the process flow of flowchart 300 proceeds to operation 335 and ends.

The process flow illustrated in FIG. 3 can be performed as a wholistic process, in which all auditory sensitivities are evaluated. Alternatively, the process flow of flowchart 300 can be performed separately for different auditory sensitivities. For example, if operation 310 results in the generation or a neural health map, different expected or estimated auditory sensitivity thresholds can be determined for different portions of the recipient's cochlea. Accordingly, the determination of operation 325 can be specific to different areas of the cochlea and/or different frequencies of sound. Accordingly, a recipient's high frequency auditory sensitivity as determined in operation 320 can fail to meet or exceed a predicted or expected high frequency auditory sensitivity threshold. As a result, the process flow of flowchart 300 can proceed to operation 330 for the recipient's high frequency auditory sensitivity, prescribing auditory training intended to improve the recipient's high frequency sensitivity. Concurrently, the recipient's low frequency auditory sensitivity determined in operation 320 can meet or exceed the predicted or expected low frequency auditory threshold in operation 325. Accordingly, the process flow can conclude for low frequency auditory sensitivity, proceeding to operation 335. Similar separate implementations of the process flow of flowchart 300 can be implemented for different hearing characteristics, such as separate processing for phoneme discrimination, word emphasis recognition, speech recognition, and other characteristics of recipient hearing known to the skilled artisan.

As noted above, operation 310 can include the generation of a neural health map for a recipient. Using a neural health map constructed from NRT thresholds and electrode distance, known stimulation parameters from device settings, and/or from individual recipient factors such as age and duration of deafness, auditory performance can be predicted. From such a neural health map, a performance threshold is set based on the information that is expected to be transmitted by a given pattern of neural survival, degree of focusing, and assumed spread of excitation. The determination of such a performance threshold from a neural health map can be an embodiment of operation 210 of FIG. 2, or operation 310 of FIG. 3. One specific example of this would be to create a matrix of expected phonemic confusions based on the neural map. Next, the subjective or behavioral auditory sensitivity of the recipient is measured with a behavioral hearing test that measures speech understanding or information transmission through psychophysics, such as phoneme discrimination or spectral ripple tests. Such tests can be an embodiment of operation 320 of FIG. 3. A targeted auditory training program is prescribed for the recipient by comparing the expected performance to the measured auditory sensitivity test result. An example process for generating a neural health map that can be used in a process as described above will now be described with reference to FIGS. 4 and 5.

With reference now made to FIG. 4, a description of generating a neural health map is provided. In particular, a method of neural health map generation is described for the neurons of a cochlea. The method utilizes measures of electrode placement in conjunction with NRT measurements to generate the neural health map.

Depicted in FIG. 4 are a series of electrodes 405a-c arranged relative to a complement of neurons 410, such as the neurons arranged about the modiolus of the cochlea. As explained below, the distances 420a-c between electrodes 405a-c and the neurons 410 are obtained from a physical measurement of electrode placement, such as Computed Tomography (CT), x-ray or magnetic resonance imaging of the electrodes. Additional techniques for determining electrode placement can include Electrode Voltage Tomography (EVT) techniques. The EVT measurements may be stored in a transimpedance matrix (TIM). The values stored in the TIM can be used to determine the location of the electrodes relative to the neurons of the cochlea.

Regardless of the method used to determine the distances 420a-c, the techniques of the present disclosure correlate the distances 420a-c with the stimulation signals (stimulations) 415a-c necessary to evoke a response of the complement of neurons 410 in regions 425a-c, respectively. For the purposes of the present disclosure, the illustrated magnitudes of the stimulation signals 415a-c, which are represented by the shaded regions, are generally indicative of the level/threshold of stimulation needed to evoke a response in the complement of neurons 410 within regions 425a-c, respectively.

In the example of FIG. 4, the correlation of the distances 420a-c to the stimulation signals 415a-c can be used to determine neural health within regions 425a-c, respectively. With respect to electrode 405a and the neurons 410 of region 425a, because both the estimated distance 420a from the electrode 405a to neurons 410 of region 425a and the stimulation signal 415a are low, it is determined that the neurons 410 within regions 425a have a good level of neural health. Accordingly, a neural health map for neurons 410 would indicate that the neurons within region 425a have a normal level of neural health.

With respect to electrode 405b and the neurons 410 within region 425b, the magnitude of stimulation signals 415b that is necessary to evoke a response in region 425b is larger than the magnitude of the stimulation signal 415a. The increased magnitude of stimulation 415b is not, however, an indication of poor health for the neurons arranged within region 425b. Instead, by correlating the distance and stimulation level/threshold, it is determined that electrode 405b would require increased stimulation to evoke a response in region 425b because distance 420b is greater than distance 420a, not because of decreased neural health of neurons 410 within region 425b. Accordingly, a neural health map for neurons 410 would indicate that region 425b has a normal level of neural health.

The relationship between the distances 420a and 420b from electrodes 405a and 405b to neurons 410 in regions 425a and 425b, respectively, is monotonic—as the distances 420a and 420b between electrodes 405a and 405b and neurons 410 decreases so does the magnitude of stimulation needed to evoke a response, as the distance between electrodes 405a and 405b and neurons 410 increases so does the magnitude of stimulation needed to evoke a response. Accordingly, the large stimulation 415b associated with electrode 405b is not indicative of poor neuron health within region 425b because distance 420b is also correspondingly larger. Turning to electrode 405c, the large stimulation 415c of electrode 405c, on the other hand, is indicative of poor neuron health.

Specifically, the illustrated magnitude of the stimulation signals 415c is associated with a larger magnitude of stimulation (as indicated by the larger magnitude of shaded region 415c) to evoke a response in region 425c of complement of neurons 410. Because distance 420c is not appreciably larger than distance 420a, but the magnitude of stimulation signal 415c is appreciably greater than that of stimulation signals 415a, the magnitude of stimulation signal 415c is, in fact, indicative of poor neuron health within region 425c. Similarly, if stimulation signal 415c is increased without any detected response from region 425c, this can serve as an indication of neuron death within region 425c. Accordingly, a neural health map can be determined for regions 425a-c in which regions 425a and 425b have a normal level of neural health and region 425c has a poor level of neural health.

Turning to FIG. 5, depicted therein is a neural health map 500 mapped onto a cochlea 540. The mapping provided by neural health map 500 was generated according to the techniques described herein, such as the techniques described above with regard to FIG. 4. Illustrated in FIG. 5 are the modiolar wall 520 (e.g., the wall of the scala tympani 508 adjacent the modiolus 512) and the lateral wall 518 (e.g., the wall of the scala tympani 508 positioned opposite to the modiolus 512). Also shown in FIG. 5 is a cochlea opening 542 which can be, for example, a natural opening (e.g., the round window) or a surgical opening (e.g., a cochleostomy). Cochlea 540 includes a mapping of its neural health in the form of regions 525a-e. As illustrated through its shading, region 525c has been mapped as having poor neural health, while regions 525a, 525b, 525d and 525e have been mapped as having good neural health.

Neural health map 500 in combination with a subjective or behavioral measure of a recipient's hearing will provide for the determination of a targeted auditory training recommendation for the recipient. For example, based on neural health map 500 it can be determined that predicted sensitivity thresholds for frequencies associated with regions 525a, 525b, 525d and 525e, all of which have good or normal neural health, should be lower than the predicted sensitivity threshold for frequencies associated with region 525c, which has poor neural health. Based on this neural health information, the results of a subjective or behavioral measure of a recipient's auditory sensitivity can be more accurately interpreted to provide targeted auditory training for the recipient. For example, if a recipient illustrates a low level of sensitivity in auditory frequencies associated with region 525c, this can be interpreted as being the best possible result for the recipient given the low neural health in region 525c. As a result, auditory training provided to the recipient might not include exercises designed to improve sensitivity in the frequencies associated with region 525c—even though recipient's sensitivity is low for these frequencies, training is unlikely to improve this sensitivity as region 525c has poor neural health. On the other hand, a similarly low level of sensitivity for frequencies associated with regions 525a, 525b, 525d and 525e would likely result in recommending auditory training intended to improve auditory sensitivity for the frequencies associated with these regions—these regions have good or normal health, and therefore, it would be expected that poor auditory sensitivity in the frequencies corresponding to these regions can be improved. Accordingly, the use of neural health map 500 can be used to provide more targeted auditory training—omitting training where improvement is unlikely to be achieved (i.e., at frequencies associated with region 525c) and focusing on training where improvement is likely to be achieved (i.e., at frequencies associated with regions 525a, 525b, 525d and 525e).

With reference now made to FIG. 6, depicted therein is a block diagram illustrating an example fitting system 670 configured to execute the techniques presented herein. Fitting system 670 is, in general, a computing device that comprises a plurality of interfaces/ports 678(1)-678(N), a memory 680, a processor 684, and a user interface 686. The interfaces 678(1)-678(N) can comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 6, interface 678(1) is connected to cochlear implant system 102 having components implanted in a recipient 671. Interface 678(1) can be directly connected to the cochlear implant system 102 or connected to an external device that is communication with the cochlear implant systems. Interface 678(1) can be configured to communicate with cochlear implant system 102 via a wired or wireless connection (e.g., telemetry, Bluetooth, etc.).

The user interface 686 includes one or more output devices, such as a display screen (e.g., a liquid crystal display (LCD)) and a speaker, for presentation of visual or audible information to a clinician, audiologist, or other user. The user interface 686 can also comprise one or more input devices that include, for example, a keypad, keyboard, mouse, touchscreen, etc.

The memory 680 comprises auditory ability profile management logic 681 that can be executed to generate or update a recipient's auditory ability profile 683 that is stored in the memory 680. The auditory ability profile management logic 681 can be executed to obtain the results of objective evaluations of a recipient's cognitive auditory ability from an external device, such as an imaging system, an NRT system or an EVT system (not shown in FIG. 6), via one of the other interfaces 678(2)-678(N). Accordingly, auditory ability profile management logic 681 can execute logic to obtain the objective measures utilized in the techniques disclosed herein. In certain embodiments, memory 680 also comprises subjective evaluation logic 685 that is configured to perform subjective evaluations of a recipient's cognitive auditory ability and provide the results for use by the auditory ability profile management logic 681. Accordingly, subjective evaluation logic 685 can be configured to implement or receive the subjective measures from which a behavioral auditory sensitivity is determined for recipient 671. In other embodiments, the subjective evaluation logic 685 is omitted and the auditory ability profile management logic 681 is executed to obtain the results of subjective evaluations of a recipient's cognitive auditory ability from an external device (not shown in FIG. 6), via one of the other interfaces 678(2)-678(N).

The memory 680 further comprises profile analysis logic 687. The profile analysis logic 687 is executed to analyze the recipient's auditory profile (i.e., the correlated results of the objective and subjective evaluations) to identify correlated stimulation parameters that are optimized for the recipient's cognitive auditory ability. Profile analysis logic 687 can also be configured to implement the techniques disclosed herein in order to generate and/or provide targeted auditory training to recipient 671 based upon the subjective and objective measures acquired by subjective evaluation logic 685 and auditory ability profile management logic 681, respectively.

Memory 680 can comprise read only memory (ROM), random access memory (RAM), magnetic disk storage media devices, optical storage media devices, flash memory devices, electrical, optical, or other physical/tangible memory storage devices. The processor 684 is, for example, a microprocessor or microcontroller that executes instructions for the auditory ability profile management logic 681, the subjective evaluation logic 685, and the profile analysis logic 687. Thus, in general, the memory 680 can comprise one or more tangible (non-transitory) computer readable storage media (e.g., a memory device) encoded with software comprising computer executable instructions and when the software is executed (by the processor 684) it is operable to perform the techniques described herein.

The correlated stimulation parameters identified through execution of the profile analysis logic 687 are sent to the cochlear implant system 102 for instantiation as the cochlear implant's current correlated stimulation parameters. However, in certain embodiments, the correlated stimulation parameters identified through execution of the profile analysis logic 687 are first displayed at the user interface 686 for further evaluation and/or adjustment by a user. As such, the user can refine the correlated stimulation parameters before the stimulation parameters are sent to the cochlear implant system 102. Similarly, the targeted auditory training provided to recipient 671 can be presented to the recipient via user interface 686. The targeted auditory training provided to recipient 671 can also be sent to an external device, such as external device 110 of FIG. 1D, for presentation to recipient 671.

As described above, the techniques of this disclosure can be implemented via the processing systems and devices of a fitting system, such as fitting system 670 of FIG. 6. According to other embodiments, a general purposes computing system or device, such as a personal computer, smart phone, or tablet computing device, can be used to implement the disclosed techniques. The disclosed techniques can also be implemented via a server or distributed computing system. For example, a fitting system, such as fitting system 670 of FIG. 6, or an external device, such as external device 110 of FIG. 1D, can transmit data including the results of objective and subjective measures to a server device or distributed computing system. Using this data, the server device or distributed computing system can implement the disclosed techniques.

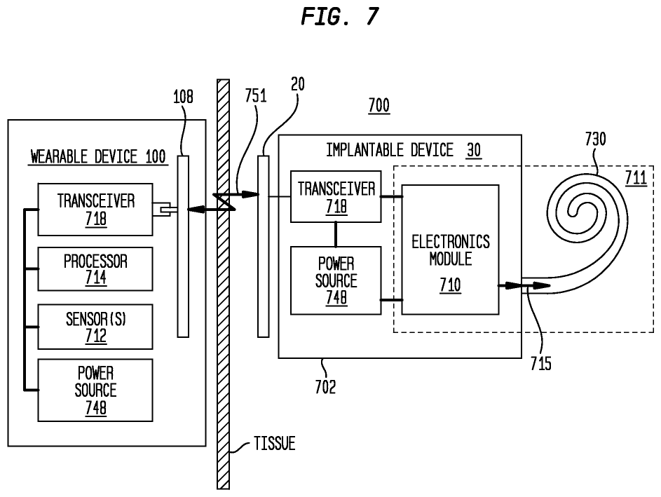

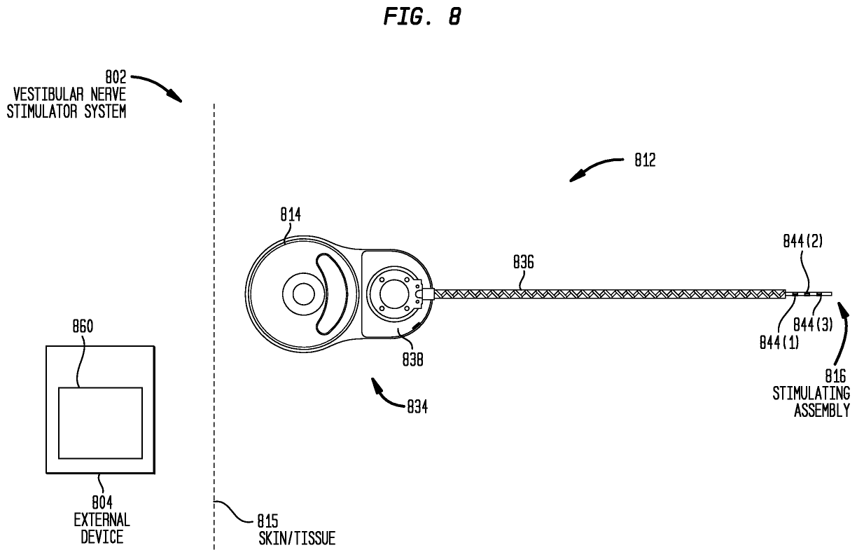

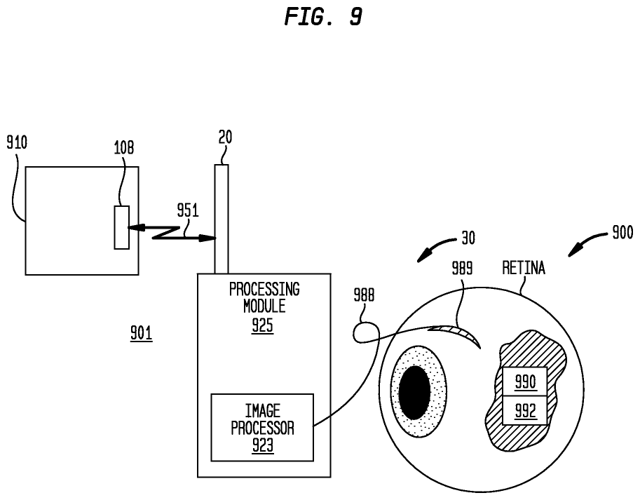

As previously described, the technology disclosed herein can be applied in any of a variety of circumstances and with a variety of different devices. Example devices that can benefit from technology disclosed herein are described in more detail in FIGS. 7-9, below. As described below, the operating parameters for the devices described with reference to FIGS. 7-9 can be configured using a fitting system analogous to fitting system 670 of FIG. 6. For example, the techniques described herein can be to prescribe recipient training for a number of different types of wearable medical devices, such as an implantable stimulation system as described in FIG. 7, a vestibular stimulator as described in FIG. 8, or a retinal prosthesis as described in FIG. 9. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 7 is a functional block diagram of an implantable stimulator system 700 that can benefit from the technologies described herein. The implantable stimulator system 700 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 702. Here, the wearable device 100 is configured to transcutaneously couple with the implantable device 30 via a wireless connection to provide additional functionality to the implantable device 30.

In the illustrated example, the wearable device 100 includes one or more sensors 712, a processor 714, a transceiver 718, and a power source 748. The one or more sensors 712 can be one or more units configured to produce data based on sensed activities. In an example where the stimulation system 700 is an auditory prosthesis system, the one or more sensors 712 include sound input sensors, such as a microphone, an electrical input for an FM hearing system, other components for receiving sound input, or combinations thereof. Where the stimulation system 700 is a visual prosthesis system, the one or more sensors 712 can include one or more cameras or other visual sensors. Where the stimulation system 700 is a cardiac stimulator, the one or more sensors 712 can include cardiac monitors. The processor 714 can be a component (e.g., a central processing unit) configured to control stimulation provided by the implantable device 30. The stimulation can be controlled based on data from the sensor 712, a stimulation schedule, or other data. Where the stimulation system 700 is an auditory prosthesis, the processor 714 can be configured to convert sound signals received from the sensor(s) 712 (e.g., acting as a sound input unit) into signals 751. The transceiver 718 is configured to send the signals 751 in the form of power signals, data signals, combinations thereof (e.g., by interleaving the signals), or other signals. The transceiver 718 can also be configured to receive power or data. Stimulation signals can be generated by the processor 714 and transmitted, using the transceiver 718, to the implantable device 30 for use in providing stimulation.

In the illustrated example, the implantable device 30 includes a transceiver 718, a power source 748, and a medical instrument 711 that includes an electronics module 710 and a stimulator assembly 730. The implantable device 30 further includes a hermetically sealed, biocompatible implantable housing 702 enclosing one or more of the components.

The electronics module 710 can include one or more other components to provide medical device functionality. In many examples, the electronics module 710 includes one or more components for receiving a signal and converting the signal into the stimulation signal 715. The electronics module 710 can further include a stimulator unit. The electronics module 710 can generate or control delivery of the stimulation signals 715 to the stimulator assembly 730. In examples, the electronics module 710 includes one or more processors (e.g., central processing units or microcontrollers) coupled to memory components (e.g., flash memory) storing instructions that when executed cause performance of an operation. In examples, the electronics module 710 generates and monitors parameters associated with generating and delivering the stimulus (e.g., output voltage, output current, or line impedance). In examples, the electronics module 710 generates a telemetry signal (e.g., a data signal) that includes telemetry data. The electronics module 710 can send the telemetry signal to the wearable device 100 or store the telemetry signal in memory for later use or retrieval.

The stimulator assembly 730 can be a component configured to provide stimulation to target tissue. In the illustrated example, the stimulator assembly 730 is an electrode assembly that includes an array of electrode contacts disposed on a lead. The lead can be disposed proximate tissue to be stimulated. Where the system 700 is a cochlear implant system, the stimulator assembly 730 can be inserted into the recipient's cochlea. The stimulator assembly 730 can be configured to deliver stimulation signals 715 (e.g., electrical stimulation signals) generated by the electronics module 710 to the cochlea to cause the recipient to experience a hearing percept. In other examples, the stimulator assembly 730 is a vibratory actuator disposed inside or outside of a housing of the implantable device 30 and configured to generate vibrations. The vibratory actuator receives the stimulation signals 715 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 718 can be components configured to transcutaneously receive and/or transmit a signal 751 (e.g., a power signal and/or a data signal). The transceiver 718 can be a collection of one or more components that form part of a transcutaneous energy or data transfer system to transfer the signal 751 between the wearable device 100 and the implantable device 30. Various types of signal transfer, such as electromagnetic, capacitive, and inductive transfer, can be used to usably receive or transmit the signal 751. The transceiver 718 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 748 can be one or more components configured to provide operational power to other components. The power source 748 can be or include one or more rechargeable batteries. Power for the batteries can be received from a source and stored in the battery. The power can then be distributed to the other components as needed for operation.

As should be appreciated, while particular components are described in conjunction with FIG. 7, technology disclosed herein can be applied in any of a variety of circumstances. The above discussion is not meant to suggest that the disclosed techniques are only suitable for implementation within systems akin to that illustrated in and described with respect to FIG. 7. In general, additional configurations can be used to practice the methods and systems herein and/or some aspects described can be excluded without departing from the methods and systems disclosed herein.

FIG. 8 illustrates an example vestibular stimulator system 802, with which embodiments presented herein can be implemented. As shown, the vestibular stimulator system 802 comprises an implantable component (vestibular stimulator) 812 and an external device/component 804 (e.g., external processing device, battery charger, remote control, etc.). The external device 804 comprises a transceiver unit 860. As such, the external device 804 is configured to transfer data (and potentially power) to the vestibular stimulator 812,

The vestibular stimulator 812 comprises an implant body (main module) 834, a lead region 836, and a stimulating assembly 816, all configured to be implanted under the skin/tissue (tissue) 815 of the recipient. The implant body 834 generally comprises a hermetically-sealed housing 838 in which RF interface circuitry, one or more rechargeable batteries, one or more processors, and a stimulator unit are disposed. The implant body 134 also includes an internal/implantable coil 814 that is generally external to the housing 838, but which is connected to the transceiver via a hermetic feedthrough (not shown).

The stimulating assembly 816 comprises a plurality of electrodes 844(1)-(3) disposed in a carrier member (e.g., a flexible silicone body). In this specific example, the stimulating assembly 816 comprises three (3) stimulation electrodes, referred to as stimulation electrodes 844(1), 844(2), and 844(3). The stimulation electrodes 844(1), 844(2), and 844(3) function as an electrical interface for delivery of electrical stimulation signals to the recipient's vestibular system.

The stimulating assembly 816 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 812, the external device 804, and/or another external device, can be configured to implement the techniques presented herein. That is, the vestibular stimulator 812, possibly in combination with the external device 804 and/or another external device, can include an evoked biological response analysis system, as described elsewhere herein.

FIG. 9 illustrates a retinal prosthesis system 901 that comprises an external device 910 (which can correspond to the wearable device 100) configured to communicate with a retinal prosthesis 900 via signals 951. The retinal prosthesis 900 comprises an implanted processing module 925 (e.g., which can correspond to the implantable device 30) and a retinal prosthesis sensor-stimulator 990 is positioned proximate the retina of a recipient. The external device 910 and the processing module 925 can communicate via coils 108, 20.

In an example, sensory inputs (e.g., photons entering the eye) are absorbed by a microelectronic array of the sensor-stimulator 990 that is hybridized to a glass piece 992 including, for example, an embedded array of microwires. The glass can have a curved surface that conforms to the inner radius of the retina. The sensor-stimulator 990 can include a microelectronic imaging device that can be made of thin silicon containing integrated circuitry that convert the incident photons to an electronic charge.

The processing module 925 includes an image processor 923 that is in signal communication with the sensor-stimulator 990 via, for example, a lead 988 which extends through surgical incision 989 formed in the eye wall. In other examples, processing module 925 is in wireless communication with the sensor-stimulator 990. The image processor 923 processes the input into the sensor-stimulator 990, and provides control signals back to the sensor-stimulator 990 so the device can provide an output to the optic nerve. That said, in an alternate example, the processing is executed by a component proximate to, or integrated with, the sensor-stimulator 990. The electric charge resulting from the conversion of the incident photons is converted to a proportional amount of electronic current which is input to a nearby retinal cell layer. The cells fire and a signal is sent to the optic nerve, thus inducing a sight perception.

The processing module 925 can be implanted in the recipient and function by communicating with the external device 910, such as a behind-the-ear unit, a pair of eyeglasses, etc. The external device 910 can include an external light/image capture device (e.g., located in/on a behind-the-ear device or a pair of glasses, etc.), while, as noted above, in some examples, the sensor-stimulator 990 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
determining, from at least one objective measure, an estimated sensory sensitivity of a recipient of a sensory device;
determining, from at least one subjective measure, a behavioral sensory sensitivity of the recipient; and
providing a sensory training recommendation based upon the estimated sensory sensitivity and the behavioral sensory sensitivity.

2. The method of claim 1, wherein determining the behavioral sensory sensitivity comprises a speech recognition test or a phoneme discrimination test.

3. The method of claim 2, wherein at least one sensory threshold value associated with the speech recognition test or the phoneme discrimination test is determined based upon the estimated sensory sensitivity.

4. The method of claim 1, wherein determining the estimated sensory sensitivity comprises determining neural health of the recipient.

5. The method of claim 1, wherein the at least one objective measure comprises at least one of:
a neural response threshold measurement;
an electroencephalogram measurement;
an electrocochleography measurement;
a blood test;
a measure of an age of the recipient; or
a measure of a length of time the recipient has experienced hearing loss.

6. The method of claim 1, wherein providing the sensory training recommendation comprises:
comparing the estimated sensory sensitivity to the behavioral sensory sensitivity; and
selecting the sensory training recommendation based on the comparing.

7. The method of claim 1, wherein determining the estimated sensory sensitivity comprises generating a neural health map for the recipient and determining the estimated sensory sensitivity from the neural health map.

8. The method of claim 7, wherein generating the neural health map comprises:
determining, for each electrode of a plurality of electrodes of the sensory device, a distance between each electrode of the plurality of electrodes and one or more neurons;
determining a stimulation threshold for each electrode of the plurality of electrodes to evoke a response in the one or more neurons;
correlating the stimulation threshold for each electrode of the plurality of electrodes with the distance between each electrode of the plurality of electrodes and the one or more neurons; and
generating a neural health map for the one or more neurons based upon the correlating the stimulation threshold for each electrode of the plurality of electrodes with the distance between each electrode of the plurality of electrodes and the one or more neurons.

9. The method of claim 1, wherein the sensory training recommendation comprises one or more of:
syllable counting training;
word emphasis training;
phoneme discrimination and identification training;
frequency discrimination training;
text following exercises;
time compressed-speech recognition exercises; or
complex speech passage comprehension exercises.

10. The method of claim 1, wherein the recipient is a recipient of a medical device, wherein the method further comprises: modifying operating parameters of the sensory device based upon the estimated sensory sensitivity and the behavioral sensory sensitivity.

11. The method of claim 1, wherein providing the sensory training recommendation comprises recommending cessation of at least one type of auditory training.

12. (canceled)

13. The method of claim 1, wherein the estimated sensory sensitivity comprises a an estimated auditory sensitivity, and wherein the behavioral sensory sensitivity comprises a behavioral auditory sensitivity.

14. (canceled)

15. (canceled)

16. (canceled)

17. (canceled)

18. (canceled)

19. (canceled)

20. (canceled)

21. One or more non-transitory computer readable storage media comprising instructions that, when executed by a processor, cause the processor to:
obtain, from at least one objective measure, an estimated sensory sensitivity of a recipient of a sensory device;
obtain a behavioral sensory sensitivity of the recipient;
determine a difference between the estimated sensory sensitivity and the behavioral sensory sensitivity; and
provide a sensory training recommendation based upon the difference between the estimated sensory sensitivity and the behavioral sensory sensitivity.

22. The non-transitory computer readable storage media of claim 21, wherein the difference between the estimated sensory sensitivity and the behavioral sensory sensitivity is less than a threshold value, and
wherein the sensory training recommendation comprises recommending cessation of at least one type of sensory training based upon the difference being less than the threshold value.

23. The non-transitory computer readable storage media of claim 21, wherein the difference between the estimated sensory sensitivity and the behavioral sensory sensitivity is greater than a threshold value, and
wherein the sensory training recommendation comprises recommending an increase of at least one type of sensory training based upon the difference being greater than the threshold value.

24. The non-transitory computer readable storage media of claim 21, further comprising instructions operable to modify operating parameters of the sensory device based upon the difference between the estimated sensory sensitivity and the behavioral sensory sensitivity.

25. The non-transitory computer readable storage media of claim 21, wherein the instructions operable to obtain the estimated sensory sensitivity comprise instructions operable to generate a neural health map for the recipient.

26. The non-transitory computer readable storage media of claim 25, wherein the instructions operable to generate the neural health map comprise instructions operable to:
determine, for each electrode of a plurality of electrodes of the sensory device, a distance between each electrode of the plurality of electrodes and one or more neurons;
determine a stimulation threshold for each electrode of the plurality of electrodes to evoke a response in the one or more neurons;
correlate the stimulation threshold for each electrode of the plurality of electrodes with the distance between each electrode of the plurality of electrodes and the one or more neurons; and
generate a neural health map for the one or more neurons based upon the correlating the stimulation threshold for each electrode of the plurality of electrodes with the distance between each electrode of the plurality of electrodes and the one or more neurons.

27. The non-transitory computer readable storage media of claim 21, wherein the sensory training recommendation comprises one or more of:
syllable counting training;
word emphasis training;
phoneme discrimination and identification training;
frequency discrimination training;
text following exercises;
time compressed-speech recognition exercises; or
complex speech passage comprehension exercises.

28. The non-transitory computer readable storage media of claim 21, wherein the instructions operable to obtain the behavioral sensory sensitivity of the recipient comprise instructions operable to obtain results of a speech recognition test or a phoneme discrimination test.

29. An apparatus comprising:
one or more memories; and
one or more processors configured to:
determine, from data stored in the one or more memories indicative of at least one objective measure, an estimated sensory sensitivity of a recipient of a sensory device;
determine, from data stored in the one or more memories indicative of at least one subjective measure, a behavioral sensory sensitivity of the recipient; and
provide a sensory training recommendation based upon the estimated sensory sensitivity and the behavioral sensory sensitivity.

30. The apparatus of claim 29, wherein the data stored in the one or more memories indicative of the at least one subjective measure comprises data indicative of results of a speech recognition test or a phoneme discrimination test.

31. The apparatus of claim 30, wherein the one or more processors are configured to determine at least one sensory threshold value associated with the speech recognition test or the phoneme discrimination test based upon the estimated sensory sensitivity.

32. The apparatus of claim 29, wherein the one or more processors are configured to determine the estimated sensory sensitivity by determining neural health of the recipient.

33. (canceled)

34. The apparatus of claim 29, wherein the one or more processors are configured to provide the sensory training recommendation by:
comparing the estimated sensory sensitivity to the behavioral sensory sensitivity; and
selecting the sensory training recommendation based on the comparing.

35. The apparatus of claim 29, wherein the one or more processors are configured to determine the estimated sensory sensitivity by generating a neural health map for the recipient and determining the estimated sensory sensitivity from the neural health map.

36. (canceled)

37. (canceled)
